(19) United States
(12) Patent Application Publication
(10) Pub. No.: US 2021/0085232 A1
(43) Pub. Date: Mar. 25, 2021

(54) FATIGUE DEGREE ESTIMATION METHOD, FATIGUE DEGREE ESTIMATION DEVICE AND PROGRAM

(71) Applicant: Nippon Telegraph and Telephone Corporation, Tokyo (JP)

(72) Inventors: Nobuaki Matsuura, Tokyo (JP); Yuichi Higuchi, Tokyo (JP); Hiroyoshi Togo, Tokyo (JP)

(21) Appl. No.: 16/978,002

(22) PCT Filed: Feb. 18, 2019
(86) PCT No.: PCT/JP2019/005766

§ 371 Date: Sep. 3, 2020

(30) Foreign Application Priority Data
Mar. 7, 2018 (JP) 2018-040678

(51) Int. Cl. A61B 5/16; A61B 5/0456; A61B 5/0452; G16H 50/30

(52) U.S. Cl. CPC A61B 5/165; G16H 50/30; A61B 5/04525; A61B 5/0456

(57) ABSTRACT

The fatigue degree estimation device includes: an R-wave detection unit that detects an R-wave from an electrocardiogram waveform of a subject; an R-R interval calculation unit that calculates an R-R interval which is a time interval between the R-wave detected by the R-wave detection unit and an immediately preceding R-wave; a difference calculation unit that calculates a difference between the R-R intervals away from each other by a certain heart rate; a percentage calculation unit that calculates a percentage of an absolute value of the difference between the R-R intervals exceeding a certain value in a target period of fatigue degree estimation; and a fatigue degree estimation unit that estimates a fatigue degree of the subject based on the percentage.

### CROSS-REFERENCE TO RELATED APPLICATIONS

This application is a national phase entry of PCT Application No. PCT/JP2019/005766, filed on Feb. 18, 2019 which claims priority to Japanese Patent Application No. 2018-040678, filed on Mar. 7, 2018, which applications are hereby incorporated herein by reference.

### TECHNICAL FIELD

The present invention relates to a fatigue degree estimation method of estimating a fatigue degree of a human from a heart rate variability, a fatigue degree estimation device, and a program.

### BACKGROUND

In recent years, wearable heartbeat measurement devices have been developed, and heartbeats have been easily monitored in various scenes.

A heartbeat interval varies under the influence of an autonomic nerve. A function of the autonomic nerve is evaluated by analysis of heart rate variability.

According to Non-Patent Literature 1, it is known that fatigue during exercise is fatigue of the brain, specifically, of a central part of the autonomic nerve. It is considered that fatigue of the central part of the autonomic nerve has an effect on heart rate variability.

The heart rate variability is analyzed using an index of a frequency domain such as LF (Low Frequency)/HF (Hi Frequency), CVRR (coefficient of variation of R-R interval which is an interval between an R-wave of electrocardiogram and an immediately preceding R-wave), and an index of a time domain such as RR50.

When a fatigue degree of a human can be estimated by monitoring of heart rate variability, such information can be utilized in scenes such as sports for individuals and teams.

However, the analysis in the frequency domain is generally not stable in accuracy and is difficult to grasp a clear tendency unless data is obtained in a significantly controlled environment. Further, even in the analysis of the time domain, the CVRR may be easily affected by mixing of artifacts due to, for example, a body motion.

FIGS. 11 and 12 are diagram showing time-series data of an R-R interval when the same person is taking a break in the middle of climbing on different days. FIGS. 11 and 12 show data for 5 minutes, respectively. It can be seen that a respiratory heart rate variability reflecting the function of the autonomic nerve of the climber appears in the example of FIG. 11. On the other hand, only a small amount of the heart rate variability appears in the example of FIG. 12.

In other words, it is considered that a state indicated by the time-series data of the R-R interval in FIG. 11 is a state in which the fatigue degree of the climber is small, and a state indicated by the data in FIG. 12 is a state in which the fatigue degree of the climber is large. For such data, when RR50 (a percentage of the difference between R-R intervals adjacent to each other exceeding 50 ms) is obtained, for example, the RR50 is 0.9% in the example of FIG. 11 and is 0% in the example of FIG. 12, which are not remarkable in difference. In order to utilize heart rate variability in estimating the fatigue degree of an exercising person, an index indicating a clear tendency is desirable, but the index indicating such a clear tendency has not been known in a conventional analysis method.

### CITATION LIST

### Non-Patent Literature

Non-Patent Literature 1: Osami KAJIMOTO, “Cause of All fatigue is Brain”, Shueisha Shinsho, p. 19-23, 2016

### SUMMARY

### Technical Problem

Embodiments of the present invention have been made in view of the above-described problems, and is to provide a fatigue degree estimation method, a fatigue degree estimation device, and a program that can obtain a clear fatigue degree estimation result from heart rate variability of a human using a simple method.

### Means for Solving the Problem

A fatigue degree estimation method according to embodiments of the present invention includes: a first step of detecting an R-wave from an electrocardiogram waveform of a subject; a second step of calculating an R-R interval which is a time interval between the R-wave detected in the first step and an immediately preceding R-wave; a third step of calculating a difference between the R-R intervals away from each other by a certain heart rate; and a fourth step of estimating a fatigue degree of the subject based on the difference between the R-R intervals.

In one configuration example of the fatigue degree estimation method according to embodiments of the present invention, the certain heart rate is any one of 6 to 9.

In one configuration example of the fatigue degree estimation method according to embodiments of the present invention, the method further includes, between the third step and the fourth step, a fifth step of calculating a percentage of an absolute value of the difference between the R-R intervals exceeding a certain value in a target period of fatigue degree estimation, and the fourth step includes a step of estimating the fatigue degree of the subject based on the percentage.

In one configuration example of the fatigue degree estimation method according to embodiments of the present invention, the fourth step includes a step of estimating that the fatigue degree of the subject is large when the percentage is equal to or less than a threshold and estimating that the fatigue degree of the subject is small when the percentage exceeds the threshold.

A fatigue degree estimation device according to embodiments of the present invention includes: an R-wave detection unit that detects an R-wave from an electrocardiogram waveform of a subject; an R-R interval calculation unit that calculates an R-R interval which is a time interval between the R-wave detected by the R-wave detection unit and an immediately preceding R-wave; a difference calculation unit that calculates a difference between the R-R intervals away from each other by a certain heart rate; and a fatigue degree estimation unit that estimates a fatigue degree of the subject based on the difference between the R-R intervals.

A fatigue degree estimation program according to embodiments of the present invention causes a computer to execute: a first step of detecting an R-wave from an electrocardiogram waveform of a subject; a second step of calculating an R-R interval which is a time interval between the R-wave detected in the first step and an immediately preceding R-wave; a third step of calculating a difference between the R-R intervals away from each other by a certain heart rate; and a fourth step of estimating a fatigue degree of the subject based on the difference between the R-R intervals.

### Effects of Embodiments of the Invention

According to embodiments of the present invention, it is possible to index respiratory heart rate variability and to obtain a clear fatigue degree estimation result with a simple method by detecting an R-wave from an electrocardiogram waveform of a subject, calculating an R-R interval which is a time interval between the detected R-wave and an immediately preceding R-wave, and calculating a difference between R-R intervals apart by a certain heart rate from each other.

### BRIEF DESCRIPTION OF THE DRAWINGS

FIG. 1 is a diagram showing an example of data of a difference between R-R intervals adjacent to each other.

FIG. 2 is a diagram showing another example of data of a difference between R-R intervals adjacent to each other.

FIG. 3 is a diagram showing an example of data of R-R intervals away from each other by 6 heartbeats.

FIG. 4 is a diagram showing another example of data of R-R intervals away from each other by 6 heartbeats.

FIG. 5 is a diagram showing a relation between a heart rate, which is a time-axis interval between R-R intervals for calculating a difference, and a percentage of an absolute value of a difference between the calculated R-R intervals exceeding 50 ms.

FIG. 6 is a block diagram showing a configuration of a fatigue degree estimation device according to an embodiment of the present invention.

FIG. 7 is a flowchart illustrating an operation of the fatigue degree estimation device according to the embodiment of the present invention.

FIG. 8 is a block diagram showing a configuration of an R-wave detection unit of the fatigue degree estimation device according to the embodiment of the present invention.

FIG. 9 is a flowchart illustrating an operation of the R-wave detection unit of the fatigue degree estimation device according to the embodiment of the present invention.

FIG. 10 is a block diagram showing a configuration example of a computer that realizes the fatigue degree estimation device according to the embodiment of the present invention.

FIG. 11 is a diagram showing an example of time-series data of an R-R interval.

FIG. 12 is a diagram showing another example of time-series data of an R-R interval.

### DETAILED DESCRIPTION OF ILLUSTRATIVE EMBODIMENTS

### Principle of Embodiments of Invention

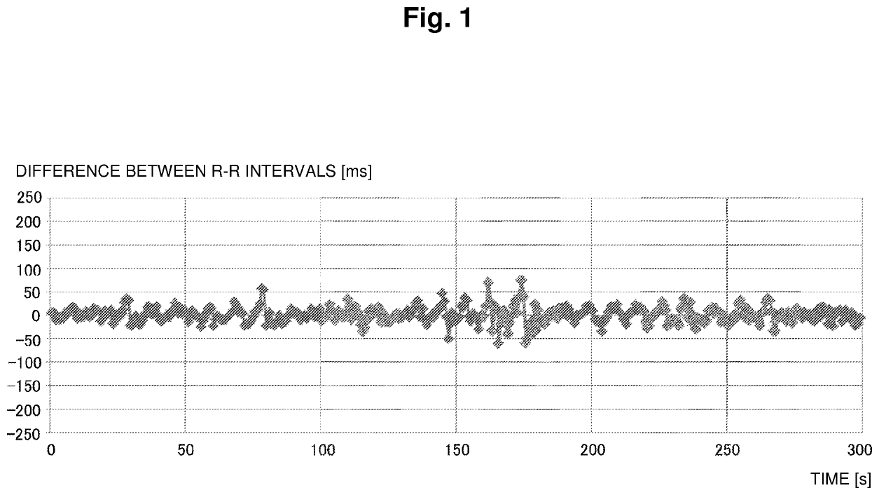

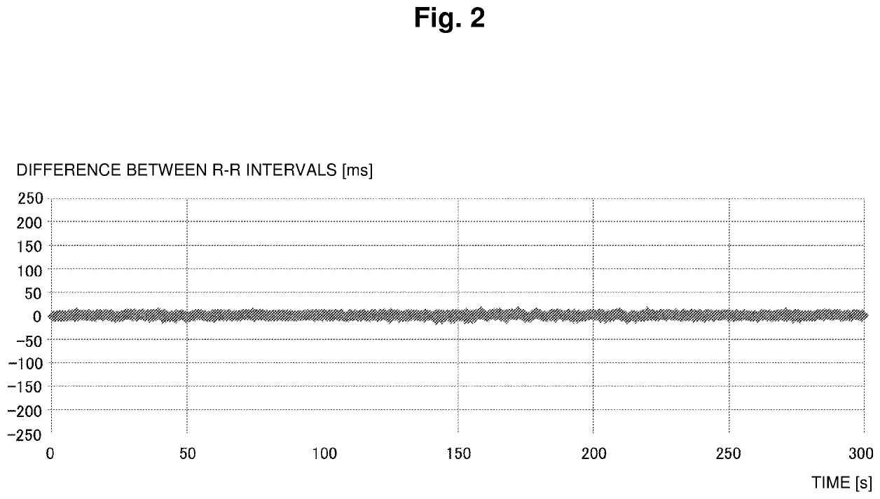

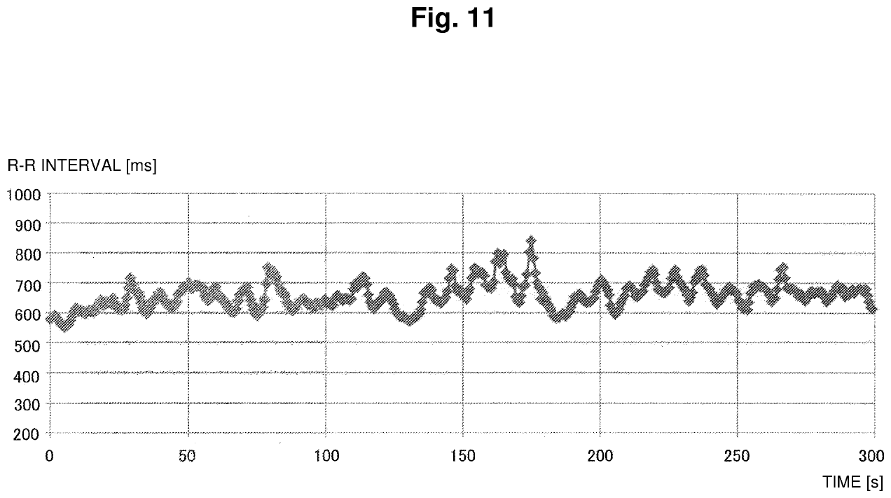

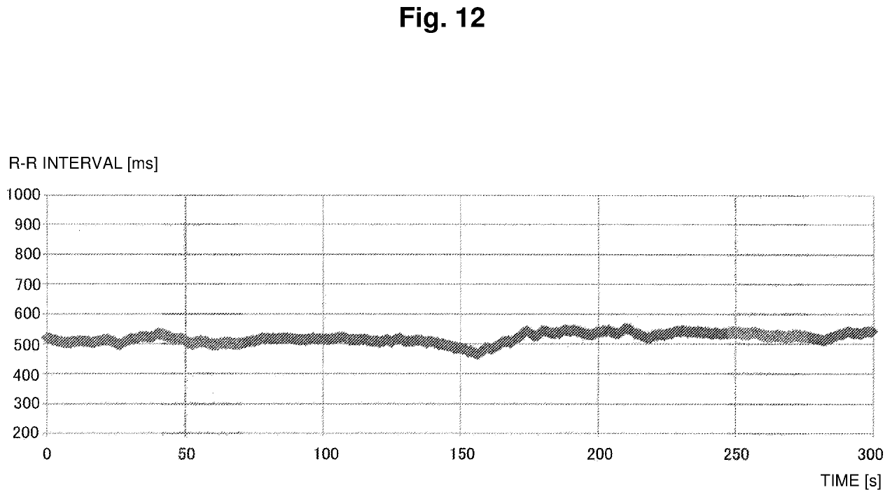

FIGS. 1 and 2 are plots of difference between R-R intervals adjacent to each other in time-series data at R-R intervals in FIGS. 11 and 12, respectively. In an example of FIG. 1, the influence of respiratory heart rate variability is seen, while in an example of FIG. 2, almost smooth fluctuation characteristics are seen. In FIG. 1, variations in a value due to the heart rate variability exist, but the absolute value of the difference in R-R intervals hardly exceeds 50 ms. In the time-series data of the R-R intervals shown in FIG. 11, which is an origin FIG. 1, the data is data of a climber during a rest period, and since the heart rate is around 90 bpm and the heart rate during one breath is large, the absolute value of the difference between R-R intervals adjacent to each other hardly exceeds 50 ms. Accordingly, the value of RR50 is extremely small in any of the examples of FIG. 1 (or FIG. 11) and FIG. 2 (or FIG. 12), and there is no difference.

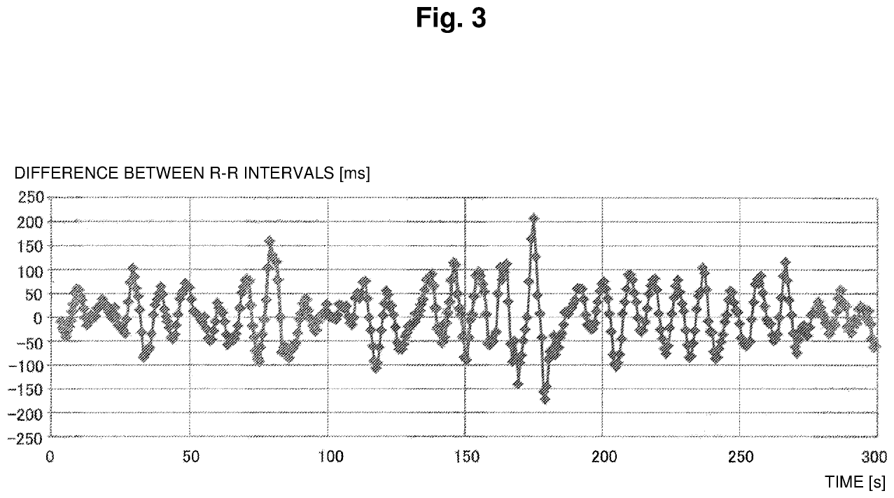

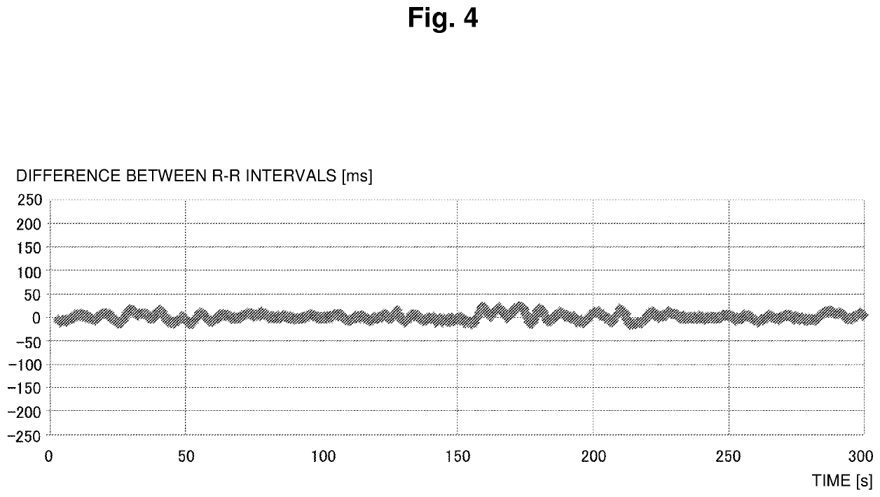

R intervals away by 6 heartbeats in time-series data at R-R intervals in FIGS. 11 and 12, respectively. Note that a value of the R-R interval at any point on FIGS. 11 and 12 is a time interval between a time of an R-wave (heartbeat time) at that point and a time of an immediately preceding (before one heartbeat) R-wave (heartbeat time), and a value at any point on FIGS. 3 and 4 is a difference between an R-R interval at that point and an R-R interval ahead of six intervals (appearing before six heartbeats).

In the example of FIG. 3, the range of variation in the difference between the R-R intervals is widened, but in the example of FIG. 4, the variation is still small. In other words, in the example of FIG. 3, the change in the difference between the R-R intervals becomes apparent because the difference between the R-R intervals away from each other by 6 heartbeats matches the cycle of increase and decrease of the R-R interval due to a breath. On the other hand, in the example of FIG. 4, since the influence of respiratory heart rate variability does not appear in the original data of FIG. 12, the amount of change in the difference between the R-R intervals away from each other by 6 heartbeats is small.

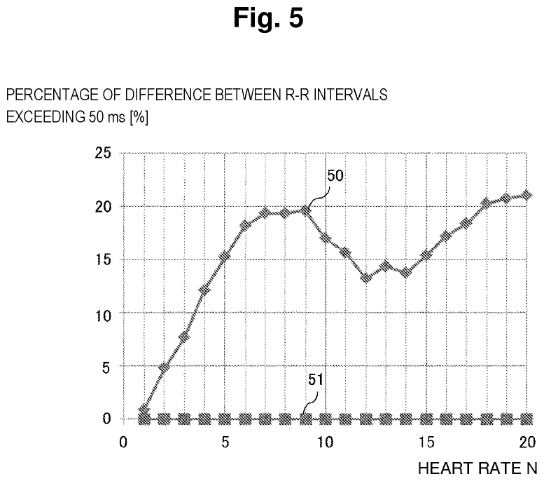

FIG. 5 is a diagram showing a relation between heart rates N, which are a time-axis interval between R-R intervals for calculating a difference, and a percentage of an absolute value of a difference between the calculated R-R intervals exceeding 50 ms. Numerical value 50 indicated in FIG. 5 represents a value calculated from the time-series data of the R-R interval in FIG. 11, and numerical value 51 represents a value calculated from the time-series data of the R-R interval in FIG. 12.

In the case of calculation from the time-series data of the R-R interval in FIG. 12, even when the heart rate N is increased, the percentage of the absolute value of the difference between the R-R intervals exceeding 50 ms remains at 0%.

On the other hand, in the case of calculation from the time-series data of the R-R interval in FIG. 11, the percentage of the absolute value of the difference between the R-R intervals exceeding 50 ms rises significantly as the heart rate N increases. Such a percentage is about 20% and reaches almost a peak value when the heart rate N is 6 to 9, and falls thereafter. Further, the percentage of the absolute value of the difference between the R-R intervals exceeding 50 ms rises again after falling. The rising reflects fluctuations in the R-R interval associated with the next breath, but may occur due to the lapse of more time and fluctuation factors other than the breath.

As described above, it is understood that when the heart rate N is 6 to 9, the fluctuation factors other than the breath can be excluded as much as possible, and an index indicating clearly the difference between the case of FIG. 11 and the case of FIG. 12 can be obtained. The reason for the difference in the percentage of the absolute value of the difference between the R-R intervals exceeding 50 ms is thought to be due to a relation between the heart rate and the breathing rhythm at that time, but mainly due to the emphasis on a respiratory heart rate variability in a single breath when the heart rate N is about 6 to 9. In this case, it can be estimated that a fatigue degree of a human is large when the percentage of the absolute value of the difference between the R-R intervals exceeding 50 ms is close to 0% the fatigue degree is small when the percentage is ten-and-several % or more.

### Embodiment

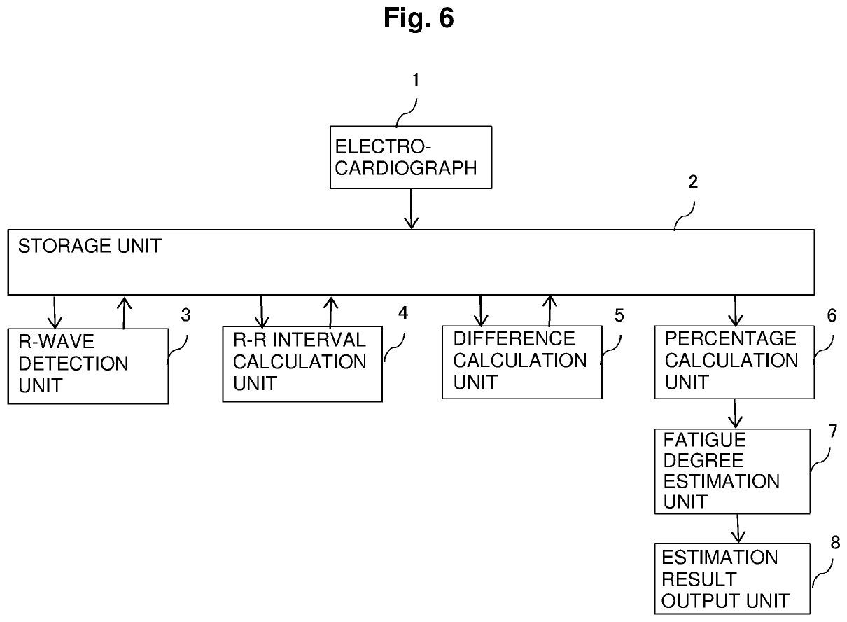

An embodiment of the present invention will be described below with reference to the drawings. FIG. 6 is a block diagram showing a configuration of a fatigue degree estimation device according to the embodiment of the present invention. The fatigue degree estimation device includes the following eight units.

An electrocardiograph 1 outputs a sampling data row of an ECG (Electrocardiogram) waveform.

A storage unit 2 stores the sampling data row of the ECG waveform and information of a sampling time.

An R-wave detection unit 3 detects an R-wave from the sampling data row of the ECG waveform.

An R-R interval calculation unit 4 calculates an R-R interval from time-series data corresponding to a time of the R-wave.

A difference calculation unit 5 calculates a difference between R-R intervals, which are away from each other by a certain heart rate, for each R-R interval.

A percentage calculation unit 6 calculates a percentage of an absolute value of a difference between R-R intervals exceeding a certain value in a target period of fatigue degree estimation.

A fatigue degree estimation unit 7 estimates a fatigue degree of a subject based on the calculated percentage.

An estimation result output unit 8 outputs estimation results.

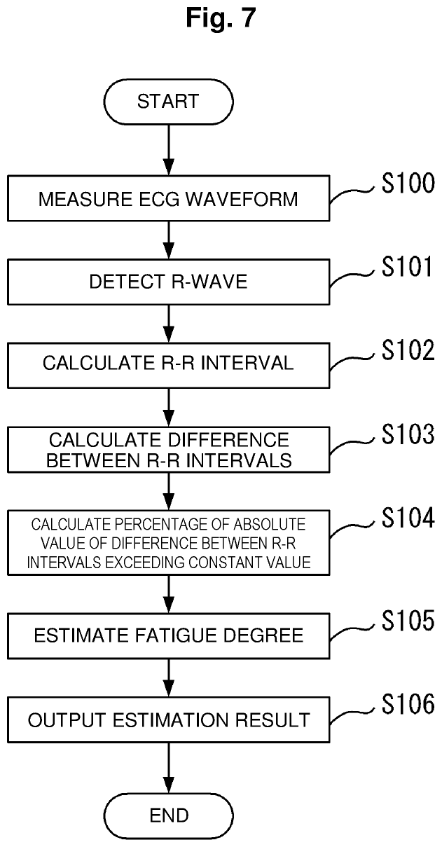

An operation of the fatigue degree estimation device according to the embodiment will be described below with reference to FIG. 7. In the embodiment, a data row obtained by sampling the ECG waveform is defined as D(i). A number i (i=1, 2, . . . ) represents a number assigned to one sampling data. It goes without saying that the greater the number i, the later the sampling time.

The electrocardiograph 1 measures the ECG waveform of a subject whose fatigue degree is to be estimated, and outputs the sampling data row D(i) of the ECG waveform (step S100 in FIG. 7). At this time, the electrocardiograph 1 adds information on the sampling time to each sampling data and outputs the sampling data. Note that a specific method of measuring the ECG waveform is a well-known technique and a detailed description thereof will be omitted. The storage unit 2 stores the sampling data row D(i) of the ECG waveform and the information on the sampling time which are output from the electrocardiograph 1.

As is well known, the ECG waveform is formed from continuous heartbeat waveforms, and one heartbeat waveform is formed from components such as P, Q, R, S, and T waves reflecting the activities of atriums and ventricles.

The R-wave detection unit 3 detects an R-wave from the sampling data row D(i) of the ECG waveform stored in the storage unit 2 (step S101 in FIG. 7).

When the ECG waveform is acquired using the wearable electrocardiograph 1 during measurement of the ECG waveform, noise accompanying body motion or the like is likely to be mixed. Such mixing of noise may cause an R-wave detection error. In particular, sudden vibration of the baseline of the ECG waveform may be erroneously detected as an R-wave. Therefore, the inventors have proposed a method capable of accurately detecting an R-wave (heartbeats) even from ECG waveform data having baseline vibration (Japanese Patent Application No. 2017-076622). The R-wave detection unit 3 will be described below based on the proposed method.

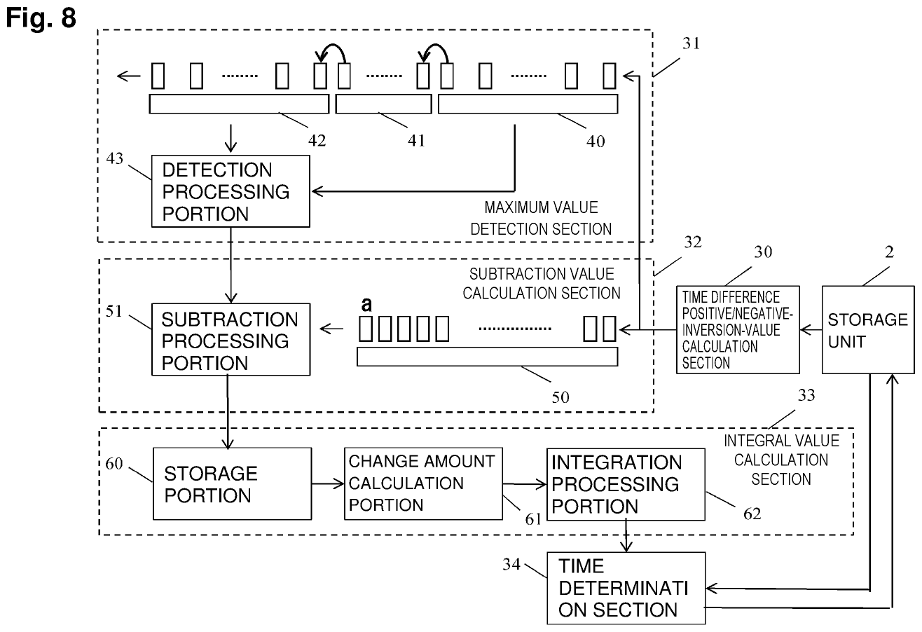

FIG. 8 is a block diagram showing a configuration of the R-wave detection unit 3. The R-wave detection unit 3 includes the following portions.

A time difference positive/negative-inversion-value calculation section 30 calculates, every sampling time, a positive/negative inversion value of time difference of sampling data from a sampling data row of the ECG waveform.

A maximum value detection section 31 detects, every sampling time, a maximum value out of positive/negative inversion values in a constant time range before a sampling time of a processing object and positive/negative inversion values in a constant time range after a sampling time of a processing object.

A subtraction value calculation section 32 calculates, every sampling time, a subtraction value obtained by subtracting the maximum value from the positive/negative inversion value of the sampling time of the processing object.

An integral value calculation section 33 calculates, every sampling time, the amount of change in the subtraction value in a range from the latest subtraction value calculated for the sampling time of the processing object to a subtraction value at a times before a predetermined time, and integrates the amount of change.

A time determination section 34 determines the sampling time of the processing object as an R-wave time (heartbeat time) when the integral value exceeds a predetermined threshold.

The maximum value detection section 31 includes FIFO (First In, First Out) buffers and a detection processing portion which will be described below. A FIFO buffer 40 receives the time difference positive/negative inversion value calculated by the time difference positive/negative-inversion-value calculation section 30 as an input. A FIFO buffer 41 receives an output value of the FIFO buffer 40 as an input. A FIFO buffer 42 receives an output value of the FIFO buffer 41 as an input. A detection processing portion 43 detects, every sampling time, a maximum value out of time difference positive/negative inversion values stored in the FIFO buffer 40 and time difference positive/negative inversion values stored in the FIFO buffer 42.

The subtraction value calculation section 32 includes a FIFO buffer 50 that receives the time difference positive/negative inversion value calculated by the time difference positive/negative-inversion-value calculation section 30 as an input and a subtraction processing portion 51 that calculates, every sampling time, a subtraction value obtained by subtracting the maximum value detected by the maximum value detection section 31 from the output value of the FIFO buffer 50.

The integral value calculation section 33 includes a storage portion 60 that stores the subtraction value calculated by the subtraction processing portion 51, a change amount calculation portion 61 that calculates, every sampling time, the amount of change in the subtraction value in a range from the latest subtraction value to the subtraction value at a time before a predetermined time, and an integration processing portion 62 that integrates the amount of change of the subtraction value in the range from the latest subtraction value to the subtraction value at a time before a predetermined time.

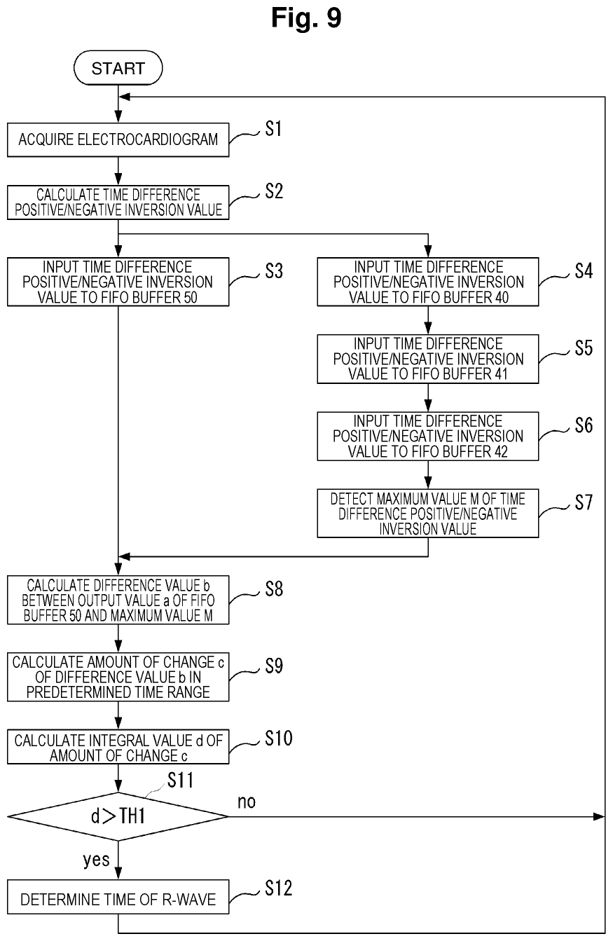

A method of detecting the R-wave according to the embodiment will be described below with reference to FIG. 9. In the description, a procedure from detection of one R-wave (heartbeat) to acquisition of the time of the R-wave will be described. Time-series data of the time of the R-wave can be obtained by repetitive calculation of such a time over the period of the ECG waveform data.

The time difference positive/negative-inversion-value calculation section 30 acquires data D(i+1) after one sampling of sampling data D(i) and data D(i−1) before one sampling of sampling data D(i), from the storage unit 2, so as to calculate a time difference positive/negative inversion value Y(i) of the sampling data D(i) (step S1 in FIG. 9). Then, the time difference positive/negative-inversion-value calculation section 30 calculates the time difference positive/negative inversion value Y(i) of the sampling data D(i) every sampling time as in the following formula (step S2 in FIG. 9).

Y(i)=−{D(i+1)−D(i−1)} (1)

The time difference positive/negative-inversion-value calculation section 30 inputs the calculated time difference positive/negative inversion value Y(i) to the FIFO buffer 50 every sampling time (step S3 FIG. 9). The input value is retained in the FIFO buffer 50 and is used for subtraction processing after a time corresponding the size of the FIFO buffer 50 (a delay time until being output from when the time difference positive/negative inversion value is input to the FIFO buffer 50).

In addition, the time difference positive/negative-inversion-value calculation section 30 inputs the calculated time difference positive/negative inversion value Y(i) to the FIFO buffer 40 every sampling time (step S4 in FIG. 9). The value output from the FIFO buffer 40 is input to the FIFO buffer 41 (step S5 in FIG. 9), and the value output from the FIFO buffer 41 is input to the FIFO buffer 42 (step S6 in FIG. 9). The FIFO buffers 40 to 42 are used for obtaining the maximum value of the time difference positive/negative inversion value in a certain time range.

A time interval L3 (a delay time until being output from when the time difference positive/negative inversion value is input to the FIFO buffer 41) corresponding to the size of the FIFO buffer 41 needs to be sufficiently wide with respect to a width (about 10 ms) of a peak derived from the R-wave, and is preferably about 50 ms. Further, a time interval L2 (a delay time until being output from when the time difference positive/negative inversion value is input to the FIFO buffer 40) corresponding to the size of the FIFO buffer 40 and a time interval L4 (a delay time until being output from when the time difference positive/negative inversion value is input to the FIFO buffer 42, L2=L4) corresponding to the size of the FIFO buffer 42 are suitably about 100 ms. The time interval L1 corresponding to the size of the FIFO buffer 50 may be L1=L2+L3/2. Therefore, the time interval L1 is 125 ms in the above numerical example. By the relation of L1=L2+L3/2 and L2=L4, a maximum value M can be obtained in a range from −(L2+L3/2) to −(L3/2) and a range from (L3/2) to (L2+L3/2) with respect to a time (a sampling time of the processing object) of an output value “a” of the FIFO buffer 50, and the maximum value M can be subtracted from the output value “a”.

The detection processing portion 43 detects, every sampling time, the maximum value M of the time difference positive/negative inversion value stored in the FIFO buffer 40 and the time difference positive/negative inversion value in the FIFO buffer 42 (step S7 in FIG. 9).

The subtraction processing portion 51 calculates a subtraction value b (=a−M) obtained by subtracting the maximum value M from the output value “a” of the FIFO buffer 50, every sampling time (step S8 FIG. 9). The subtraction value “b” calculated by the subtraction processing portion 51 is stored in the storage portion 60.

The change amount calculation portion 61 calculates the amount of change c(i) of a subtraction value b(i) calculated by the subtraction processing portion 51 with respect to a subtraction value b(i−1) before one sampling as in the following formula (step S9 in FIG. 9).

c(i)=b(i)−b(i−1) (2)

The change amount calculation portion 61 calculates, using the value stored in the storage portion 60, the amount of change “c” expressed by Formula (2) in a range from the latest subtraction value b(i) calculated by the subtraction processing portion 51 to a subtraction value b(i−N−1) before a predetermined time (20 ms in the embodiment) (N is the number of subtraction values “b” included in the range from the latest time to the predetermined time), every sampling time.

The integration processing portion 62 integrates the amounts of change c(i), c(i−1), c(i−2), and c(i−N−1) calculated every sampling time in the range from the latest subtraction value b(i) to the subtraction value b(i−N−1) before a predetermined time by the change amount calculation portion 61, as in the following formula (step S10 in FIG. 9).

d(i)=c(i)+c(i−1)+c(i−2)++c(i−N−1) (3)

However, when the amounts of change c(i), c(i−1), c(i−2), and c(i−N−1) to be integrated include a decreasing amount having a negative sign, the integration processing portion 62 calculates a value d(i) by excluding the decreasing amount from the integration and integrating only the amount of change “c” of an increasing amount having a positive sign.

The time determination section 34 determines a sampling time of the integral value d(i) as the time of the R-wave (heartbeat) when the integral value d(i) exceeds a predetermined threshold TH1 (yes in step S11 in FIG. 9) (step S12 in FIG. 9).

The integral value d(i) is obtained, as a sampling time of the processing object, the sampling time of the time difference positive/negative inversion value (output value a) ahead of time difference positive/negative inversion value, which is calculated by the time difference positive/negative-inversion-value calculation section 30, by the time interval L1. Information on the sampling time of the output value “a” can be acquired from the storage unit 2.

In this way, the time-series data of the time of the R-wave can be obtained when the processes of steps S1 to S12 are repeatedly executed every sampling cycle. The detected time-series data of the time of the R-wave is stored in the storage unit 2.

The method of detecting the R-wave described above is an example, and the R-wave may be detected by another method.

Subsequently, the R-R interval calculation unit 4 calculates, for each R-wave (for each heartbeat), an R-R interval which is a time interval between the R-wave and the immediately preceding R-wave, from the time-series data of the time of the R-wave stored in the storage unit 2 (step S102 in FIG. 7). The time-series data of the calculated R-R interval is stored in the storage unit 2.

The difference calculation unit 5 calculates a difference Dif between R-R intervals away from each other by a certain heart rate (a certain number), for each R-R interval (step S103 in FIG. 7). Specifically, the difference calculation unit 5 calculates a difference Dif between an R-R interval Inew at a certain point in the time-series data and an R-R interval Iold ahead of the R-R interval Inew by a certain heart rate CN (CN is a specific value, which is any one of 6 to 9 in the embodiment), as in the following formula.

Dif=Inew−Iold (4)

The difference calculation unit 5 calculate such a difference Dif for all data of the R-R intervals in the target period for fatigue degree estimation (for 5 minutes in the examples of FIGS. 11 and 12). However, it goes without saying that, in data of the CN R-R intervals at the beginning of the target period, the R-R interval Iold becomes 0 when no data of an R-R interval ahead of the CN R-R intervals by a certain heart rate CN. Time-series data of a difference between the calculated R-R intervals is stored in the storage unit 2.

Subsequently, the percentage calculation unit 6 calculates a percentage “r” of the absolute value of the difference Dif between the R-R intervals exceeding a certain value (50 ms in the embodiment) in the target period for the fatigue degree estimation (step S104 in FIG. 7). When the total number of data of the difference Dif between the R-R intervals in the target period for the fatigue degree estimation is defined as nall and the number of times of the absolute value |Dif| of the difference Dif between the R-R intervals exceeding a certain value in the target period for the fatigue degree estimation is defined as n, the percentage r is as follows.

r=n/nall×100[%] (5)

The fatigue degree estimation unit 7 compares the percentage r calculated by the percentage calculation unit 6 with a predetermined threshold TH2, thereby estimating a fatigue degree of a subject (step S105 in FIG. 7). Specifically, the fatigue degree estimation unit 7 estimates that the fatigue degree of the subject is large when the percentage r is equal to or less than the threshold TH2 (for example, 10%), and estimates that the fatigue degree of the subject is small when the percentage r exceeds the threshold TH2. As for the threshold TH2, a value between 0% and ten-and-several % may be defined in advance as the threshold TH2, from the result of FIG. 5.

The estimation result output unit 8 outputs the estimation result obtained by the fatigue degree estimation unit 7 (step S106 in FIG. 7). An output method at this time includes, for example, a display of the estimation result, an audio output of the estimation result, and a wireless transmission of the estimation result to an external apparatus.

Thus, the clear fatigue degree estimation result can be obtained from the heart rate variability of the subject in the embodiment.

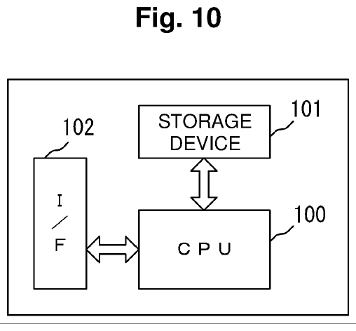

The storage unit 2, the R-wave detection unit 3, the R-R interval calculation unit 4, the difference calculation unit 5, the percentage calculation unit 6, and the fatigue degree estimation unit 7 of the fatigue degree estimation device described in the embodiment can be implemented by a computer including a CPU (Central Processing Unit), a storage device, and an interface and a program for controlling these hardware resources. A configuration example of the computer is shown in FIG. 10. The computer includes a CPU100, a storage device 101, and an interface device (hereinafter, abbreviated as I/F) 102. The electrocardiograph 1 and hardware of the estimation result output unit 8 are connected to the I/F 102. In such a computer, a fatigue degree estimation program is stored in the storage device 101 to realize the fatigue degree estimation method of embodiments of the present invention. The CPU100 executes the processing described in the embodiment according to the fatigue degree estimation program stored in the storage device 101.

### INDUSTRIAL APPLICABILITY

Embodiments of the present invention is applicable to a technique of detecting a fatigue degree of human.

### REFERENCE SIGNS LIST

1 Electrocardiograph
2 Storage unit
3 R-wave detection unit
4 R-R interval calculation unit
5 Difference calculation unit
6 Percentage calculation unit
7 Fatigue degree estimation unit
8 Estimation result output unit
30 Time difference positive/negative-inversion-value calculation section
31 Maximum value detection section
32 Subtraction value calculation section
33 Integral value calculation section
34 Time determination section
40 to 42, 50 FIFO buffer
43 Detection processing portion
51 Subtraction processing portion
61 Change amount calculation portion
62 Integration processing portion.

1.-8. (canceled)

9. A fatigue degree estimation method comprising:
detecting, by a device, a first R-wave from an electrocardiogram waveform of a subject;
calculating, by the device, a first R-R interval, the first R-R interval being a time interval between the first R-wave and a second R-wave immediately preceding the first R-wave;
calculating, by the device, a difference between the first R-R interval and a second R-R interval, wherein the first R-R interval is separated from the second R-R interval by a predetermined number of heartbeats; and
estimating, by the device, a fatigue degree of the subject based on the difference between the first R-R interval and the second R-R interval.

10. The fatigue degree estimation method according to claim 9, wherein the predetermined number of heartbeats is in a range of 6 to 9.

11. The fatigue degree estimation method according to claim 9, further comprising:
calculating a plurality of differences between a plurality of first R-R intervals and a plurality of second R-R intervals within a target period of fatigue degree estimation, wherein first R-R interval is one of the plurality of first R-R intervals and the second R-R interval is one of the plurality of second R-R intervals; and
after calculating the difference between the first R-R interval and the second R-R interval, calculating a percentage of times that an absolute value of the plurality of differences between the plurality of first R-R intervals and the plurality of second R-R intervals exceeds a certain value, wherein estimating the fatigue degree of the subject comprises estimating the fatigue degree of the subject based on the percentage.

12. The fatigue degree estimation method according to claim 11, wherein estimating the fatigue degree of the subject based on the percentage comprises:
estimating that the fatigue degree of the subject is large when the percentage is equal to or less than a threshold; and
estimating that the fatigue degree of the subject is small when the percentage exceeds the threshold.

13. A fatigue degree estimation device comprising:
a R-wave detector that detects a first R-wave from an electrocardiogram waveform of a subject;
a R-R interval calculator that calculates a first R-R interval, the first R-R interval being a time interval between the first R-wave and a second R-wave immediately preceding first R-wave;
a difference calculator that calculates a difference between the first R-R interval and a second R-R interval, the first R-R interval being separated from the second R-R interval by a predetermined number of heartbeats; and
a fatigue degree estimator that estimates a fatigue degree of the subject based on the difference between the first R-R interval and the second R-R interval.

14. The fatigue degree estimation device according to claim 13, wherein the predetermined number of heartbeats is in a range of 6 to 9.

15. The fatigue degree estimation device according to claim 13, wherein the difference calculator calculates a plurality of differences between a plurality of first R-R intervals and a plurality of second R-R intervals within a target period of fatigue degree estimation, wherein first R-R interval is one of the plurality of first R-R intervals and the second R-R interval is one of the plurality of second R-R intervals, and wherein the fatigue degree estimation device further comprises:
a percentage calculator that calculates a percentage of times an absolute value of the plurality of differences between the plurality of first R-R intervals and the plurality of second R-R intervals exceeds a certain value, wherein the fatigue degree estimator estimates the fatigue degree of the subject based on the percentage.

16. A non-transitory computer-readable media storing computer instructions for fatigue degree estimation, that when executed by one or more processors, cause the one or more processors to:
detect a first R-wave from an electrocardiogram waveform of a subject;
calculate a first R-R interval, the first R-R interval being a time interval between the first R-wave and a second R-wave immediately preceding the first R-wave;
calculate a difference between the first R-R interval and a second R-R interval, wherein the first R-R interval is separated from the second R-R interval by a predetermined number of heartbeats; and
estimate a fatigue degree of the subject based on the difference between the first R-R interval and the second R-R interval.

17. The non-transitory computer-readable media of claim 16, wherein the predetermined number of heartbeats is in a range of 6 to 9.

18. The non-transitory computer-readable media of claim 16, wherein the instructions for fatigue degree estimation, that when executed by the one or more processors, further cause the one or more processors to:
calculate a plurality of differences between a plurality of first R-R intervals and a plurality of second R-R intervals within a target period of fatigue degree estimation, wherein first R-R interval is one of the plurality of first R-R intervals and the second R-R interval is one of the plurality of second R-R intervals; and
after calculating the difference between the first R-R interval and the second R-R interval, calculate a percentage of times that an absolute value of the plurality of differences between the plurality of first R-R intervals and the plurality of second R-R intervals exceeds a certain value, wherein estimating the fatigue degree of the subject comprises estimating the fatigue degree of the subject based on the percentage.
